(19) United States
(12) Patent Application Publication
(10) Pub. No.: US 2019/0125693 A1
(43) Pub. Date: May 2, 2019

(54) KINESIOLOGY TAPE HAVING CANNABIS-DERIVED COMPOUNDS

(71) Applicant: Brandon Jones, Gilbert, AZ (US)

(72) Inventor: Brandon Jones, Gilbert, AZ (US)

(21) Appl. No.: 15/795,001

(22) Filed: Oct. 26, 2017

(51) Int. Cl. A61K 9/70; A61N 2/00; A61N 2/06; A61M 37/00; A61K 36/185

(52) U.S. Cl. CPC A61K 9/7076; A61N 2/002; A61N 1/36021; A61M 37/0015; A61K 36/185; A61N 2/06

(57) ABSTRACT

A therapeutic kinesiology tape includes a thin stretchable, elastic substrate having a first side and a second side, and an active layer disposed on the first side of the elastic substrate. The active layer has an adhesive characteristic and includes a cannabis-based mixture. In an embodiment, the cannabis-based mixture is delivered through the skin via a plurality of small-needle arrays. The kinesiology tape is available in continuous rolls and also pre-cut strips. Additionally, permanent magnets (e.g., healing magnets, acupressure magnets) may be added to the tape.

### BACKGROUND OF THE INVENTION

### 1. Field of the Invention

The present invention relates to kinesiology tape having one or more cannabis-derived compounds for delivery through the skin.

### 2. Description of the Related Art

Conventional kinesiology tape comprises a thin strip of elastic cotton adhesive tape that is applied over one or more portions of the skin to rehabilitate sore and injured muscles. The tape does not contain any medication, and is about as thick and elastic as human skin. The tape is believed to work by gently lifting the skin, which decompresses the space between the skin and the underlying tissue promoting improved blood and lymphatic flow to the injured area.

The original kinesiology tape was developed by Japanese chiropractor Kenzo Kase in the 1970's, and given the trade name Kinesio Tape®. Other brands of kinesiology tape include KT Tape®, RockTape®, and PerformTex™. Kinesiology tape is available in continuous rolls and also pre-cut strips (with “I,” “X” and “Y” shapes being the most popular). Various woven patterns, textures, designs and colors are available. Although kinesiology tape has been around for many years, substantial innovation in the tape has been largely limited to improvements in the adhesives, manufacturing methods, and ornamental designs of the tape.

### SUMMARY OF THE INVENTION

One aspect of the disclosure relates to a therapeutic kinesiology tape comprising a thin stretchable, elastic substrate having a first side and a second side; and an active layer disposed on the first side of the elastic substrate, the active layer including a cannabis-based mixture. The substrate will preferably be made substantially of fibers of cotton or hemp, or a hemp-cotton blend, and elastic fibers that that provide the material with the ability to stretch along the longitudinal axis by as much as about 20-40% of its original length. The active layer includes an adhesive or the like. The cannabis-based mixture can be applied in a pattern, such as a wave pattern or the like, disposed within grooves formed by the pattern. Alternatively, the cannabis-based mixture can have an adhesive characteristic. Preferably, in each case, the adhesive will be a pressure-sensitive adhesive. Further, to protect the adhesive from the environment, a release liner can be disposed on top of the active layer.

The thickness of the therapeutic kinesiology tape will preferably be about the same thickness as human skin. When applied onto the skin over an injured area, with the active layer touching the skin, the therapeutic kinesiology tape adheres to the skin and starts delivering the cannabis-based mixture transdermally until it is absorbed by blood vessels into the bloodstream. The cannabis-based mixture can include various plant oils, terpenes, and other herbal remedies depending on such factors as the nature of the injury and efficacy of the ingredients. Additionally, permanent magnets (e.g., healing magnets, acupressure magnets) may be added to the active layer.

According to other aspects of the invention, the therapeutic kinesiology tape can alternatively or additionally include small needle delivery of all or a portion of the cannabis-based mixture. Such small needle delivery can include micro-needles or nano-needles which have substantial advantages over transdermal delivery methods. Whereas transdermal delivery methods can resist delivery through the stratum corneum, small-needles are able to easily penetrate the stratum corneum. In some embodiments, small-needle arrays can be disposed onto the active layer by placement into cavities formed in the active layer. The small-needles can be provided as micro-needle arrays, nano-needle arrays, or the like. Either hollow or solid needles can be used.

### BRIEF DESCRIPTION OF THE DRAWINGS

FIG. 1 illustrates a top view of a kinesiology tape infused with a cannabis-derived compound, according to an embodiment.

FIG. 2 illustrates a side view of the kinesiology tape of FIG. 1.

FIG. 3 illustrates a top view of a kinesiology tape infused with a cannabis-derived compound, according to another embodiment.

FIG. 4 illustrates a side view of the kinesiology tape of FIG. 3.

FIG. 5 illustrates a top view of a kinesiology tape with small-needles to deliver a cannabis-derived compound, according to an embodiment.

FIG. 6 illustrates a side view of the kinesiology tape of FIG. 5.

FIG. 7 illustrates a top view of a kinesiology tape with small-needles to deliver a cannabis-derived compound, according to another embodiment.

FIG. 8 illustrates a side view of the kinesiology tape of FIG. 7.

### DETAILED DESCRIPTION OF THE INVENTION

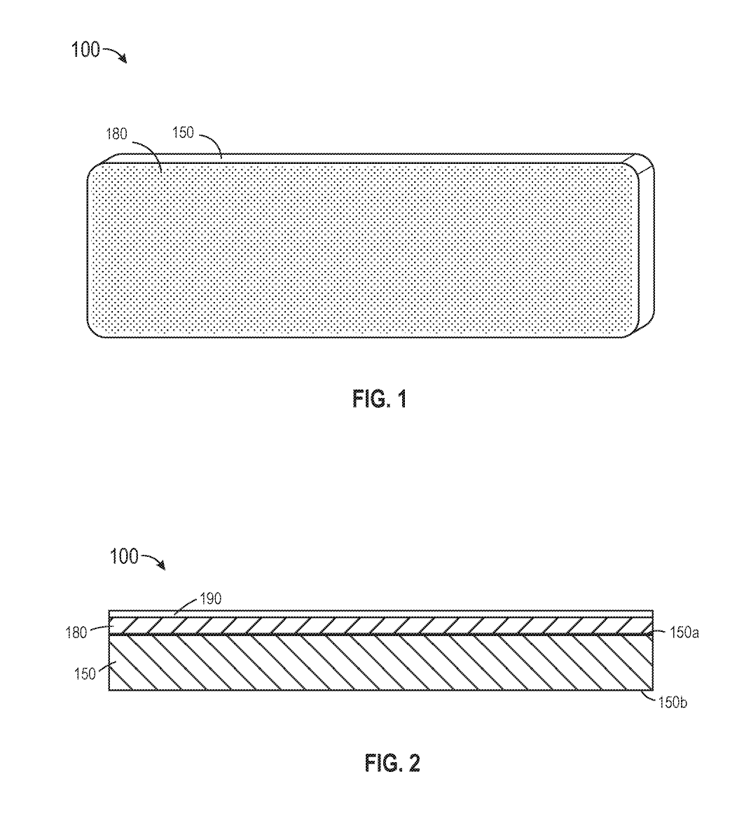

Referring to FIG. 1, a top view of a kinesiology tape 100 infused with a cannabis-derived compound, according to an embodiment, is illustrated. FIG. 2 illustrates a side view of the kinesiology tape 100. Preferably, the kinesiology tape 100 will be about the same thickness as human skin. As shown, the kinesiology tape 100 comprises an elastic substrate 150 having a first side 150a and a second side 150b. The substrate 150 will preferably be made substantially of fibers of organic cotton or hemp, or an organic hemp-cotton blend, and elastic fibers that provide the material with the ability to stretch along the longitudinal axis by as much as about 20-40% of its original length. An active layer 180 is disposed on the first side 150a of the elastic substrate 150, the active layer 180 including a cannabis-based mixture that is applied to the substrate 150. The cannabis-based mixture can include various ingredients depending on the nature of the injury or ailment, as well as to provide other desired characteristics (e.g., viscosity). As a non-limiting example, the cannabis-based mixture can include a mixture of decarboxylated cannabis, aloe vera gel, vitamin E oil, and shea butter or cocoa butter. The cannabis-based mixture can also include various plant oils, terpenes, and other herbal remedies depending on such factors as the nature of the injury and efficacy of the ingredients. Additionally, permanent magnets (e.g., healing magnets, acupressure magnets) may be added to the active layer 180. The active layer 180 should have a pressure-sensitive adhesive characteristic so that the kinesiology tape 100 will stick to an affected area of skin. The active layer 180 can further include honey or agave nectar, for example, to achieve this quality. Finally, a release liner 190 is provided to protect the active layer 180 from the environment.

The kinesiology tape 100 is applied by peeling off the release liner 190, exposing the active layer 180, then gently pressing the tape 100 onto the skin over the injured area with the active layer 180 facing the skin. The kinesiology tape 100 is believed to work by gently lifting the skin, which decompresses the space between the skin and the underlying tissue promoting improved blood and lymphatic flow to the injured area. Additionally, the cannabis-based mixture which is delivered through the skin is believed to facilitate the healing process and reduce inflammation and pain.

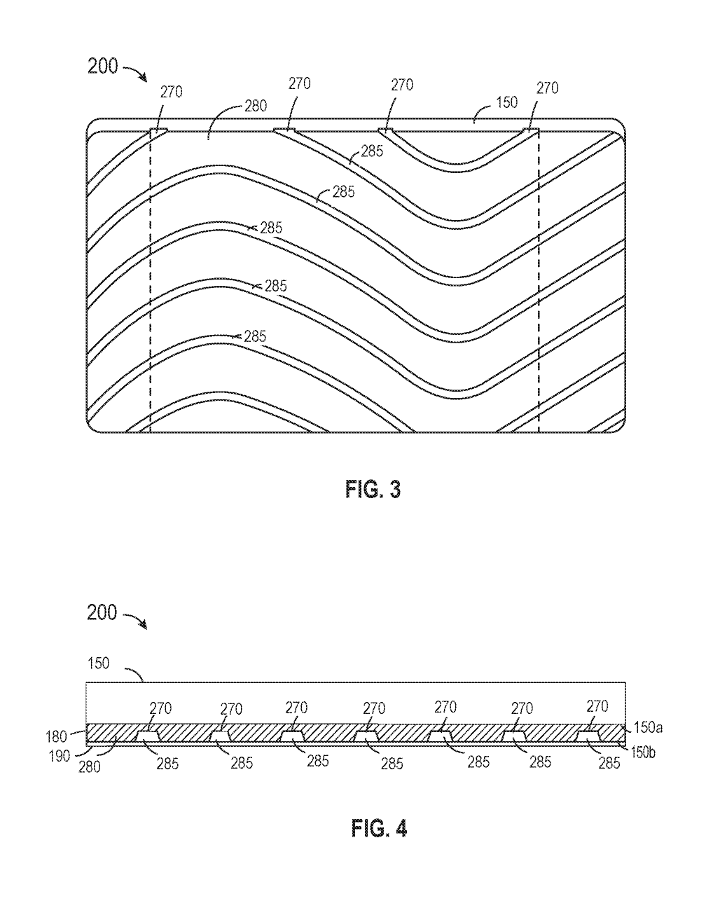

Referring to FIG. 3, a top view of a kinesiology tape 200 infused with a cannabis-derived compound, according to another embodiment, is illustrated. FIG. 4 illustrates a side view of the kinesiology tape 200. The kinesiology tape 200 is substantially the same as the kinesiology tape 100, except that the active layer 180 of the substrate 150 includes a plurality of grooves 270 wherein the cannabis-based mixture 285 is deposited. In this embodiment, the cannabis-based mixture 285 would not necessarily have adhesive elements since adhesive 280 would be deposited on the non-grooved portions of the active side 280. The adhesive 280 can include a heat-activated acrylic adhesive or the like. Although the grooves 270 are shown in a “wave” pattern, it is to be understood that other patterns and arrangements could be used.

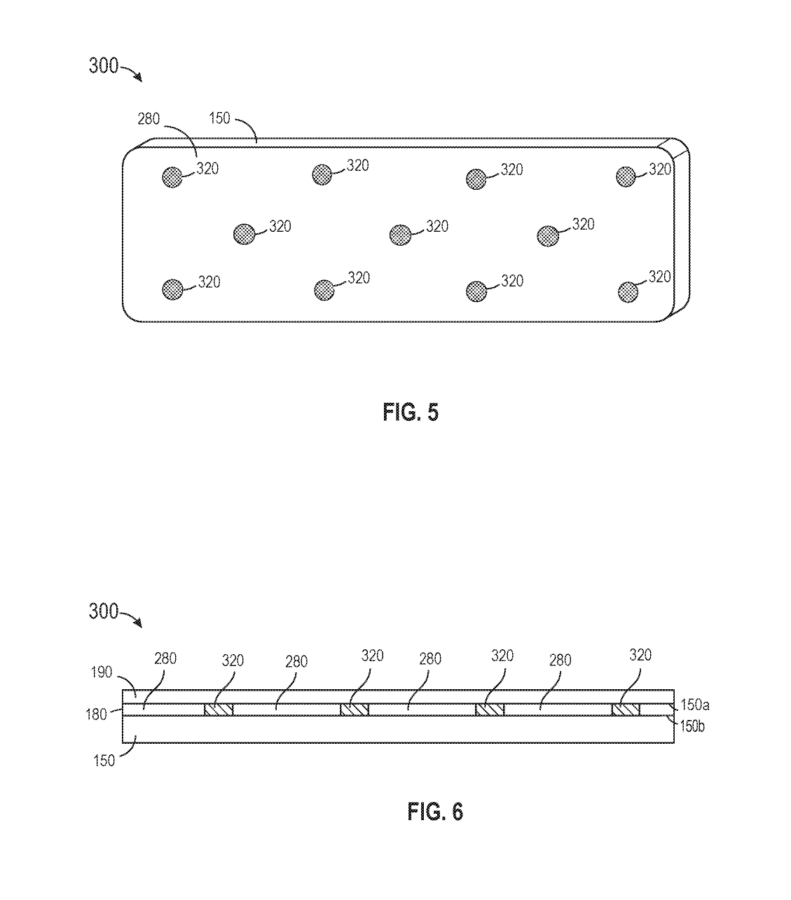

Referring to FIG. 5, a top view of a kinesiology tape 300 with small-needles to deliver a cannabis-derived compound, according to an embodiment, is illustrated. FIG. 6 illustrates a side view of the kinesiology tape 300. The kinesiology tape 300 is substantially the same as the kinesiology tape 100, except that the cannabis-based mixture is delivered using a plurality of small-needle arrays 320 that are disposed on the active side 150a of the substrate 150. A used herein, the term small-needles is defined to be micro-needles or nano-needles, as these terms are used in the art. Small-needles have substantial advantages over transdermal delivery methods. Whereas transdermal delivery methods can resist delivery through the stratum corneum, small-needles are able to easily penetrate the stratum corneum. The small-needle arrays 320 can be disposed onto the active layer 180 by placement into cavities formed in the active layer 180. Either hollow or solid small-needles can be used. In this embodiment, the cannabis-based mixture would not necessarily have adhesive elements since adhesive 280 would be deposited on the active side 180 except where the micro-needle arrays 320 are located. The adhesive 280 can include a heat-activated acrylic adhesive or the like. Although the micro-needle arrays 320 are shown as being circular, it is to be understood that other shapes, sizes and arrangements could be used.

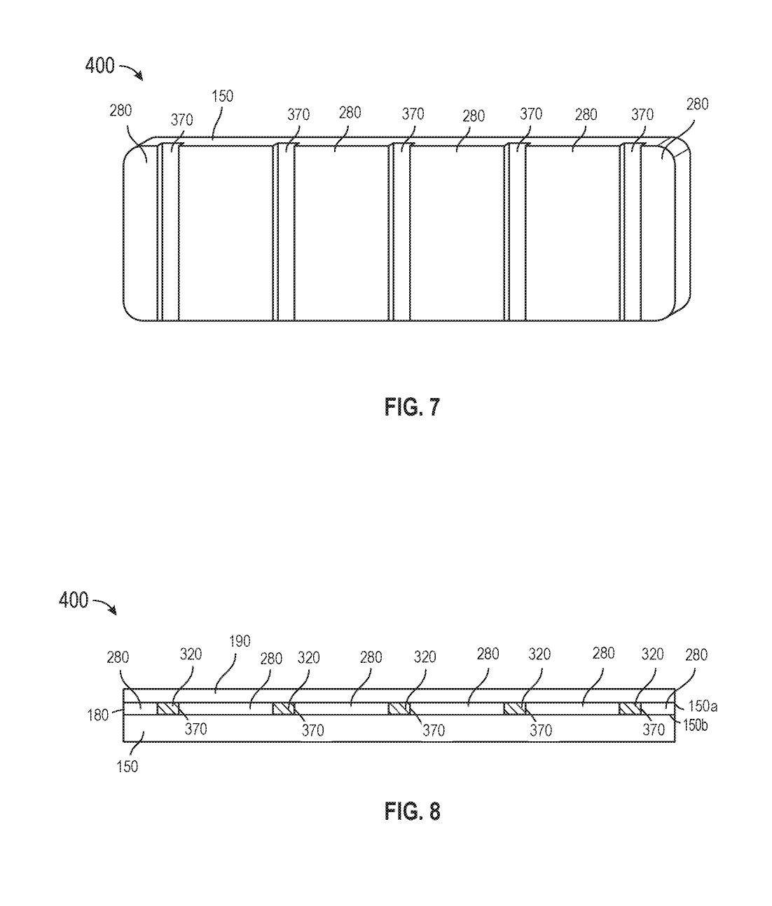

Referring to FIG. 7, a top view of a kinesiology tape 400 with small-needles to deliver a cannabis-derived compound, according to another embodiment, is illustrated. FIG. 8 illustrates a side view of the kinesiology tape 400. The kinesiology tape 400 is substantially the same as the kinesiology tape 300, except that the small-needle arrays 320 are disposed in cavities 370 formed as trenches in the active-surface 150a of the substrate 150.

As shown in the drawings, the novel therapeutic kinesiology tape 100-400 is represented as a rectangular strip. However, it is to be understood that the novel therapeutic kinesiology tape of the present invention preferably will be available in continuous rolls and also pre-cut strips (e.g., “I,” “X” and “Y” shapes). Additionally, preferably, the pre-cut strips include rounded corners to reduce fraying and to allow a more comfortable and lasting fit.

Although it was mentioned above that the substrate 150 can include organic cotton or a cotton/hemp blend, it is to be understood that non-organic or even synthetic fibers like polyester and nylon as well as certain biomaterials inspired by nature can be used in some embodiments. In general any suitable ratio, combination or composition of material is within the scope of this invention. Furthermore, in some cases, the adhesives of the present invention can include adhesives like acrylic or silicone with cannabinoid compounds applied, integrated, suspended, or otherwise merged into the fabric. In another more sophisticated example, a cannabinoid adhesive matrix is provided with terpenes, a permeation enhancer(s), carrying agent(s). In some cases, a known elemental compound like copper, silver, gold etc., can be applied to the skin facing side in order to expedite transdermal delivery while also promoting an environment that proactively protects the skin thereby facilitating an environment conducive to wound healing. In preferred embodiments, latex-free, hypoallergenic and water repellency are also important characteristics of this invention. Some embodiments will have a specialized gentle adhesive for elderly patients or for those with fragile skin.

In certain embodiments, the present invention can be a wound aid dressing. In this case, the substrate 150 need not be a kinesiology tape but could include a non-elastic material such as merely a cotton or hemp/cotton blend. In this case, the wound aid dressing could include a substrate having, for example, a cannabinoid, a pressure sensitive adhesive, and medical-grade honey. An elemental compound like copper silver or gold could be applied to the cloth on the skin-facing side in order to expedite transdermal delivery while also promoting an environment that proactively protects the skin thereby facilitating an environment conducive to wound healing.

In still other embodiments, the present invention may comprise a pliable cannabinoid-derived medicinal fabric (tapes, patches, cloths) united with a more rigid component which, in some embodiments, is provided in rolls. The medicinal fabric could include an active layer with terpenes, pressure-sensitive adhesives, and a cannabinoid-derived compound. Permeation enhancers and carrying agents could further be included to realize the transdermal qualities of the cannabinoids. Using the nasal cavity as an example, the elastic components of the tape would assist the more rigid component by distracting, decompressing, and elevating the skin thereby causing an increase in tissue permeability. Synergistically, these mechanics coalesce to lift the dermis and to expand nasal structures for enhanced breathing and respiration naturally using the terpenes, cannabinoids and other cannabinoid compounds.

Further, in another non-limiting example, the therapeutic kinesiology tape can extend beyond the nasal cavity onto the facial structures as a continuous single strip extending laterally. Yet in other embodiments, isolated strips of cannabis adhesive tape with the appropriate elastic characteristics and structural qualities is placed inferior to the ocular structures bilaterally then applied or tensioned appropriately. As in other embodiments, the substrate 150 may be enhanced by incorporating materials that permit for anti-glare properties. Pertaining to this embodiment, materials that permit the absorption, reflection or refraction of light waves, for example, may be used. In another embodiment, an insect repellent or the like can be used to deter insects or other pests.

In some embodiments, the therapeutic kinesiology tape may be designed to provide more superficial or topical relief as opposed to transdermal relief. In this instance, salves and balms might be appropriate to the end user. In other embodiments, cannabis resin, rosin or its derivatives constitute a medicated self-adhesive cannabis bandage with terpenes and bioactives.

In yet another embodiment, cannabinoids are adhered to a foam type component while in others a reservoir configuration is useful. It is to be understood that these additional components should be protected prior to application during the resting state. Moreover, when the tape is elongated or unidirectionally stretched beyond its resting state, these structures must have qualities that are resilient and capable of protecting the cannabis containing structure while being activated or extended as described.

It is to be understood that the invention described in this disclosure may not necessarily require being stretched, extended or manipulated in any way in order to be activated. Moreover, activation of the cannabinoids should not be confused necessarily with activation of the base cloth. Further activation may occur through external mechanisms or due to structural qualities inherent in the substrate.

Expanding upon this example, rare earth magnets or magnetic healing can be integrated into the fabric. In some embodiments said magnets may interact with charged or uncharged particles or other structures. In yet another embodiment, transcutaneous electrical nerve stimulation (TENS) and/or neuromuscular electrical stimulation (NMES) can be incorporated into the base cloth for healing purposes when appropriate. Those versed in the art are aware of benefits of electrophysiologic currents for the treatment of chronic wounds.

In other embodiments, a time-based delivery system is used while in other embodiments the intrinsic permeability of the base cloth is used to permeate skin layers which offers substantial advantages when compared to the prior art. While in other embodiments, a sustained release mechanism may be more appropriate to the end user. Of course, there are innumerable delivery mechanisms that could allow for absorption through the skin and into the tissues and organs of an end user. As such, those skilled in the art realize that the embodiments described herein are not all-inclusive and nor are they meant to be limiting in scope.

Cannabinoid based-derivatives and bioactives with the aforementioned characteristics can be applied onto the fabric of the substrate 150 using rollers. In some embodiments, these materials are incorporated into the base cloth during the heating process, while in other embodiments integrating during the cooling or curing process. Additional methods of manufacture include suspension, infusion, injection, uniform distribution, etc. Additional non-limiting examples include rubbing, smearing, misting, heating, cooling, cooking, spraying, submerging, baking, etc.

Similarly, in a less pliable more porous embodiment, elastic stretch fibers can be replaced with gauze or any other natural or synthetic fiber. Further bioactives having known anti-bacterial, anti-microbial, anti-viral, or even anti-fungal properties may be added as indicated in order to enhance patient outcomes.

In another embodiment, the therapeutic kinesiology tape can be engineered in such a way to provide protection against visible and invisible environmental threats like insects (as mentioned), sunlight or radiation exposure especially during athletic pursuits or activities of daily living thereby decreasing the chance of unwanted interactions between end users with other creatures or environmental objects.

Therefore, in some embodiments the base fabric can reflect, refract, absorb light waves or made to otherwise deter environmental objects. As such, in addition to the aforementioned anti-glare characteristics, in some embodiments the fabric glows in the dark or otherwise provides visual stimuli which alert and orient others during exercise activities such as jogging, for example. For instance, the invention can be designed in such a way to alert or orient other members of the environment to the presence of a moving object during activities of daily living. Finally, the tape might be adapted using a communication method such as Braille in order to alert and orient end users with functional impairments to key areas and features of the invention.

Finally, although the focus of the present disclosure has been for application of a therapeutic kinesiology tape for humans, it is to be understood that the therapeutic kinesiology tape may additionally be suitable for other animal species, such as horses.

While this invention has been described in conjunction with the various exemplary embodiments outlined above, it is evident that many alternatives, modifications and variations will be apparent to those skilled in the art. Accordingly, the exemplary embodiments of the invention, as set forth above, are intended to be illustrative, not limiting. Various changes may be made without departing from the spirit and scope of the invention.

What is claimed is:

1. A therapeutic kinesiology tape, comprising:
a thin stretchable, elastic substrate having a first side and a second side; and
an active layer disposed on the first side of the elastic substrate,
wherein the active layer has an adhesive characteristic and includes a cannabis-based mixture.

2. The therapeutic kinesiology tape of claim 1, wherein the adhesive characteristic includes being a pressure-sensitive adhesive.

3. The therapeutic kinesiology tape of claim 1, wherein the active layer includes grooves wherein the cannabis-based mixture is disposed.

4. The therapeutic kinesiology tape of claim 3, wherein an adhesive is disposed in non-grooved portions of the active layer.

5. The therapeutic kinesiology tape of claim 1, wherein the cannabis-based mixture includes terpenes.

6. The therapeutic kinesiology tape of claim 1, wherein the substrate includes hemp.

7. The therapeutic kinesiology tape of claim 6, wherein the substrate includes a hemp and cotton blend.

8. The therapeutic kinesiology tape of claim 1, wherein the active layer includes a plurality of needles capable of delivering the cannabis-based mixture through the skin of a wearer of the tape.

9. The therapeutic kinesiology tape of claim 8, wherein the needles are micro-needles.

10. The therapeutic kinesiology tape of claim 9, wherein the needles are arranged as micro-needle arrays.

11. The therapeutic kinesiology tape of claim 13, wherein the needles are hollow.
